(19) United States
(12) Patent Application Publication
(10) Pub. No.: US 2022/0071634 A1
(43) Pub. Date: Mar. 10, 2022

(54) WOUND CLOSURE SYSTEMS AND METHODS

(71) Applicant: Regents of the University of Minnesota, Minneapolis, MN (US)

(72) Inventors: Arthur G. Erdman, New Brighton, MN (US); Paul Rothweiler, Stillwater, MN (US); Aaron Paul Tucker, Saint Paul, MN (US)

(21) Appl. No.: 17/469,157

(22) Filed: Sep. 8, 2021

Related U.S. Application Data
(60) Provisional application No. 63/075,367, filed on Sep. 8, 2020.

(51) Int. Cl. A61B 17/08

(52) U.S. Cl. CPC A61B 17/085; A61M 2025/024; A61B 2017/086; A61B 2017/081

(57) ABSTRACT

A device includes a housing, a tissue engager, and a closing mechanism. The housing can be placed over a wound, and the tissue engager can engage a portion of tissue proximate the wound. The closing mechanism is coupled to the housing and the tissue engager and can move the tissue engager from an open position to a closed position to close the wound.

### PRIORITY

This application claims the benefit of the filing date of U.S. Provisional Application Ser. No. 63/075,367, filed on Sep. 8, 2020, the disclosure of which is incorporated by reference herein.

### FIELD

The present disclosure relates to wound closure devices and related methods.

### BACKGROUND

Some conventional methods for closing wounds utilize stitches or staples. However, conventional methods may be insufficient when space is limited, time is limited, all of the tools are not available, or there is not a skilled professional to perform the methods.

### BRIEF DESCRIPTION OF THE DRAWINGS

The drawings described herein are for illustrative purposes only of selected embodiments and not all possible implementations, and they are not intended to limit the scope of the present disclosure.

FIG. 1 is a top view of a wound closure device placed over a wound, in accordance with at least one example.

FIG. 2 is a cutaway top view of the wound closure device of FIG. 1 with a dual slider-crank closing mechanism in an open position, in accordance with at least one example.

FIG. 3 is a cutaway top view of the wound closure device of FIG. 2 with the dual slider-crank dosing mechanism in a dosed position, in accordance with at least one example.

FIG. 4 is a cutaway top view of a wound closure device with a single slider-crank closing mechanism in an open position, in accordance with at least one example.

FIG. 5 is a cutaway top view of the wound closure device of FIG. 4 with the single slider-crank closing mechanism in a closed position, in accordance with at least one example.

FIG. 6 is a cutaway top view of a wound closure device with a cam closing mechanism in an open position, in accordance with at least one example.

FIG. 7 is a cutaway top view of the wound closure device of FIG. 6 with the cam closing mechanism in a closed position, in accordance with at least one example.

FIG. 8 is a cutaway top view of a wound closure device with a push tab closing mechanism in an open position, in accordance with at least one example.

FIG. 9 is a cutaway top view of the wound closure device of FIG. 8 with the push tab closing mechanism in a closed position, in accordance with at least one example.

FIG. 10 is a cutaway top view of a wound closure device with an iris closing mechanism in a closed position, in accordance with at least one example.

FIG. 11 is a cutaway top view of a wound closure device with a pull closing mechanism in an open position, in accordance with at least one example.

FIG. 12 is a cutaway top view of the wound closure device of FIG. 11 with the pull closing mechanism in a closed position, in accordance with at least one example.

FIG. 13 is a cutaway top view of a wound closure device with a dosing mechanism in an open position and a tissue fastener undeployed, in accordance with at least one example.

FIG. 14 is a cutaway top view of the wound closure device of FIG. 13 with the closing mechanism in a closed position and the tissue fastener deployed, in accordance with at least one example.

FIG. 15 is a cutaway top view of the wound closure device of FIGS. 13 and 14 with the closing mechanism returned to an opened position and the tissue fastener deployed, in accordance with at least one example.

FIG. 16 is a perspective view of a wound closure device, in accordance with at least one example.

FIG. 17 is a perspective view of a wound closure device, in accordance with at least one example.

FIG. 18 is a is a flow chart of a method for closing a wound, in accordance with at least one example.

Corresponding reference numerals indicate corresponding parts throughout the several views of the drawings.

### DETAILED DESCRIPTION

A wound closure device can include a housing that houses a closing mechanism and a tissue engager. The housing can include an adhesive or other coupling mechanism for attaching to tissue (for example, skin) of a patient. The wound closure device can be placed over a wound and adhered to the tissue surrounding the wound. In some examples, the housing can include a window for viewing the wound while the housing is positioned over the wound. The tissue engager can engage a portion of tissue surrounding the wound such that movement of the tissue engager can move the portion of tissue. The closure mechanism can be actuated to move the tissue engager into a closed position to close the wound.

Sometimes there is not enough space or time for a practitioner to close a wound via suturing or stapling. Sometimes a skilled practitioner is not readily available to close the wound (for example, on a battlefield, in an emergency, etc.). The present wound closure device allows for closure of the wound while minimizing required time, skill level, and space. Further, in at least some examples, the wound closure device can be used one-handed to close the wound, freeing practitioners to address other concerns at the same time. For example, if used to close a wound following removal of an implanted tube, one hand can be used to close the wound while the other hand is removing the tube from the patient.

FIG. 1 is a top view of a wound closure device 100 placed over a wound 102, in accordance with at least one example. In the illustrated example, the wound closure device 100 is positioned about a tube 104 that is implanted in and extending from a patient's body. In at least one example, the wound closure device 100 comprises at least a portion of a tube clamp or tube anchoring device. The wound closure device 100 is positioned on tissue 106 of the patient (e.g., the skin) proximate the wound 102. The wound closure device 100 includes a housing 108, which can be any shape or form, In the illustrated example, the housing 108 includes a window 110 and a hinged portion 112. The window 110 is an opening that allows the tube 104 to extend through the housing 108. The hinged portion 112 allows the wound closure device 100 to open to receive the tube 104 and to hinge closed to clamp around the tube 104.

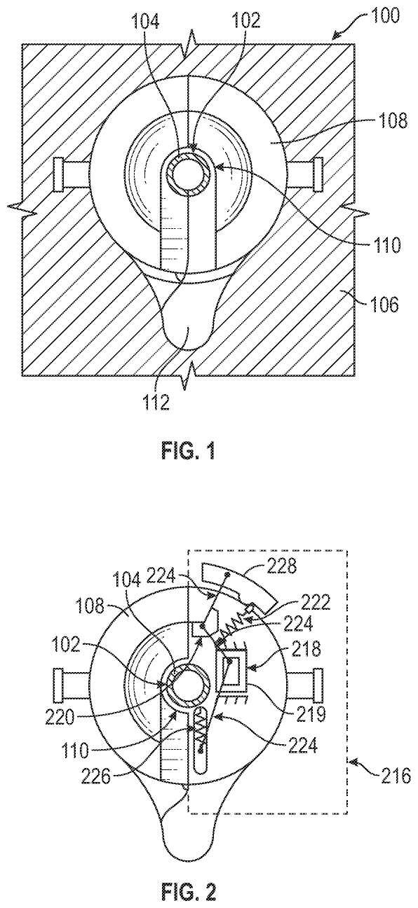

FIG. 2 is a cutaway top view of the wound closure device 100 of FIG. 1 with a dual slider-crank closing mechanism 214 in an open position, in accordance with at least one example. The cutaway plane 216 is shown with dotted lines, such that the top of the housing 108 is only cut away on one side of the wound closure device 100. A tissue engager 218 is coupled to the dual slider-crank dosing mechanism 214, such that the dosing mechanism 214 can move the tissue engager 218. A surface of the tissue engager 218 that comes in contact with the tissue 106 includes an engaging feature, for example, adhesive, hooks, teeth, grip, a combination of these, or the like to grab or otherwise engage the tissue 106. The tissue engager 218 may be smaller or larger than illustrated and may comprise any of a variety of shapes. In some examples, the tissue engager 218 includes a plurality of components to engage different portions of the tissue 106. In at least one example, the tissue engager 218 engages an adhesive pad 219 adhered to the tissue 106 instead of engaging the tissue 106 directly.

The illustrated example of a dual slider-crank closing mechanism 214 includes one or more anchors 220 (for example, pivots) coupled to the housing 108, a plurality of connectors 224, a biasing element 226, and an actuator 228. In the illustrated example, the biasing element 226 is a spring that biases the closing mechanism 214 toward the closed position. The biasing element 226 reduces the force needed for a user of the wound closure device 100 to close the closing mechanism 214. In the illustrated example, the actuator 228 is a manual actuator. In at least one example, the dual slider-crank closing mechanism 214 includes a second set of the illustrated components on the side that does not have the cutaway plane 216. In at least one example, the dual slider-crank closing mechanism 214 includes components on the side that does not have the cutaway plane 216 that are controlled by the same actuator 228. In at least one example, the dual slider-crank closing mechanism 214 includes one or more locking features 222 to overcome the bias of the biasing element 226 and lock the dual slider-crank closing mechanism 214 in the open position. In the illustrated example, the locking feature 222 includes a compression spring and tab that coordinates with a hump of the actuator 228 to keep the actuator 228 in the open position until an external force is applied to the actuator to push the hump over the locking feature 222. However, in other examples, the locking feature 222 can comprise any of a variety of locking features.

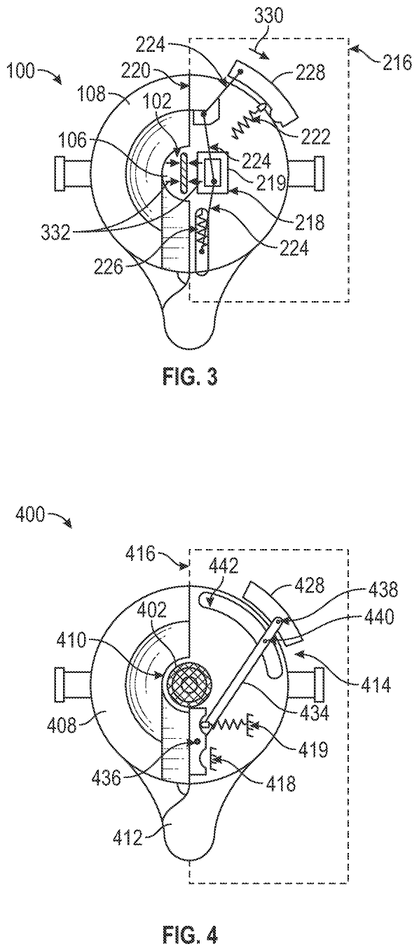

FIG. 3 is a cutaway top view of the wound closure device 100 of FIG. 2 with the dual slider-crank dosing mechanism 214 in a dosed position, in accordance with at least one example. In the illustrated example, the actuator 228 has been moved in a dosing direction 330 that (in combination with the biasing element 226) moves one or more of the connectors 224 to move the tissue engager 218 toward the wound 102 to close the wound 102. In at least one example, the tissue engager 218 pushes or pulls at least a portion of the tissue 106 to close the wound 102. In some examples, two or more tissue engagers 218 are moved toward the wound 102 or toward each other in a closing direction 332 to close the wound 102. In at least one example, the biasing element 226 maintains the tissue engager 218 in the closed position. Some examples do not require an actuator 228 to close the wound 102 and may not include an actuator 228. For example, the tube 104 may keep the tissue engager 218 in the open position, and upon removal of the tube, the biasing element 226 automatically causes the tissue engager 218 to close the wound 102.

FIG. 4 is a cutaway top view of a wound closure device 400 with a single slider-crank closing mechanism 414 in an open position, in accordance with at least one example. In the illustrated example, the wound closure device 400 includes a housing 408, a closing mechanism 414, and a tissue engager 418, The cutaway plane 416 is shown with dotted lines, such that the top of the housing 408 is only cut away on one side of the wound closure device 400. The housing 408 can be of any shape. In the illustrated example, the housing 408 includes a window 410 that allows for visibility of the wound 402 and in some examples can accommodate a tube. The illustrated example also includes a hinged portion 412, such that the wound closure device 400 can be opened and closed about the hinged portion 412, but other examples do not include the hinged portion 412.

The tissue engager 418 is coupled to the single slider-crank dosing mechanism 414, such that the dosing mechanism 414 can move the tissue engager 418. A surface of the tissue engager 418 that comes in contact with the tissue includes an engaging feature, for example, adhesive, hooks, teeth, grip, a combination of these, or the like to grab or otherwise engage the tissue. The tissue engager 418 may be smaller or larger than illustrated and may comprise any of a variety of shapes. In the illustrated example, the single slider-crank dosing mechanism 414 includes an actuator 428, a rod 434, a plurality of hinge points 436, 438, a pin 440, and a slot 442.

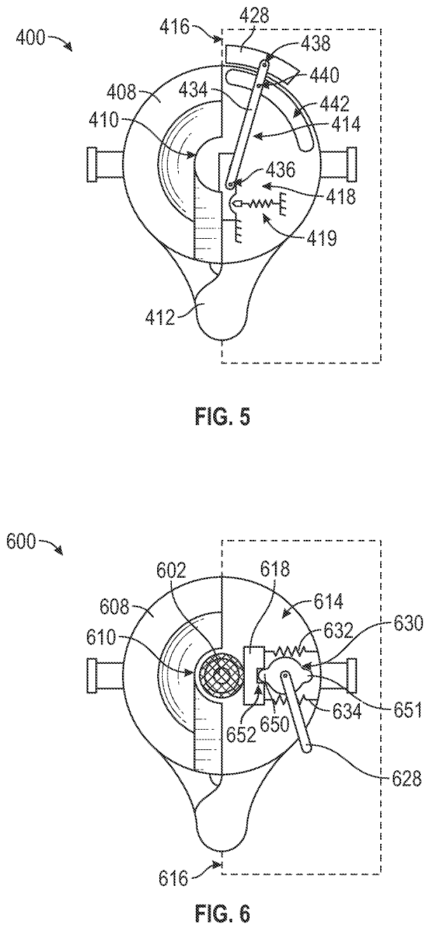

FIG. 5 is a cutaway top view of the wound closure device 400 of FIG. 4 with the single slider-crank closing mechanism 414 in a closed position, in accordance with at least one example. In the illustrated example, the actuator 428 has been moved such that the pin 440 has moved along the slot 442 relative to its position in FIG. 4. The rod 434 has pivoted about its hinge point 436 and moved the tissue engager 418 toward the wound 402 to close the wound 402. In the illustrated example, the tissue engager 418 is moved completely over the wound 402, causing a portion of the tissue to cover the wound 402. As closing mechanism 414 is moved toward the closed position, the actuator 428 rotates about a hinge point 438. In some examples, the single slider-crank closing mechanism 414 includes a locking feature 419. In at least one example the locking feature 419 can lock the single slider-crank closing mechanism 414 in an open position to prevent premature closure of the wound. In at least one example, the locking feature 419 can lock the single slider-crank closing mechanism in the closed position to prevent reopening of the closing mechanism 414 and exposure of the wound. In the illustrated example, the locking feature includes a compression spring and a tab that coordinates with a hump of the tissue engager 418, such that the locking feature 419 can lock the closing mechanism 414 in the open position and in the closed position, and a force applied to the actuator 428 can overcome the locking feature 419 to move from the open position to the dosed position or from the dosed position to the open position. In other examples, the locking feature 419 can comprise any of a variety of locking mechanisms.

FIG. 6 is a cutaway top view of a wound closure device 600 with a cam dosing mechanism 614 in an open position, in accordance with at least one example. In the illustrated example, the wound closure device 600 includes a housing 608, a closing mechanism 614, and a tissue engager 618. The cutaway plane 616 is shown with dotted lines, such that the top of the housing 608 is only cut away on one side of the wound closure device 600. The housing 608 can be of any shape. In the illustrated example, the housing 608 includes a window 610 that allows for visibility of the wound 602 and in some examples can accommodate a tube. Some examples may not include a window 610.

The closing mechanism 614 is coupled to the housing 608 and the tissue engager 618, such that the closing mechanism 614 can move the tissue engager 618. A surface of the tissue engager 618 that comes in contact with the tissue includes an engaging feature, for example, adhesive, hooks, teeth, grip, a combination of these, or the like to grab or otherwise engage the tissue. The tissue engager 618 may be smaller or larger than illustrated and may comprise any of a variety of shapes.

In the illustrated example, the cam closing mechanism 614 includes an actuator 628, a cam 630, and biasing elements 632, 634. In the illustrated example, the biasing elements 632, 634 are compression springs coupled to the tissue engager 618 such that they bias the tissue engager 618 toward the wound 602 to assist the cam 630 and reduce the force required for the actuator 628. In other examples, more or less biasing elements 632, 634 may be used. In at least one example, the cam 630 includes a locking feature 650 corresponding to a locking feature 652 of the skin engager 618 to lock the closing mechanism 614 in the open position. In some examples, the cam 630 includes a locking feature 651 corresponding to the locking feature 62 of the skin engager 618 to lock the closing mechanism 614 in the dosed position. An external force applied to the actuator 628 can cause either of the locking features 650, 651 when engaged with locking feature 652 to become unengaged. In other examples, the cam 630 may include any of a variety of locking features.

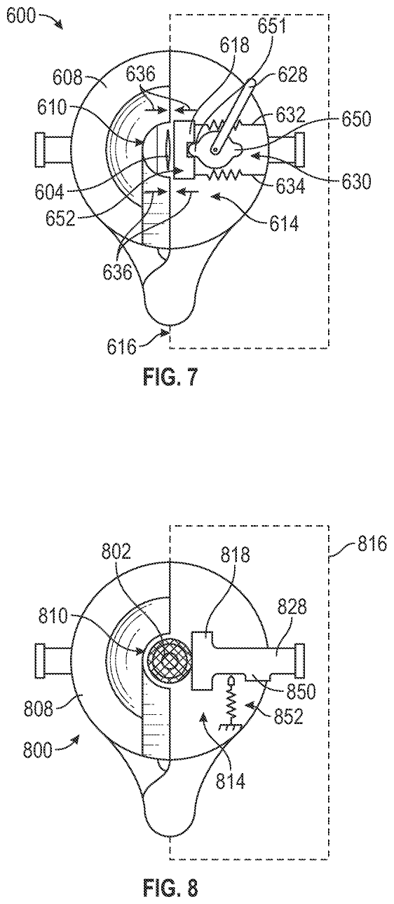

FIG. 7 is a cutaway top view of the wound closure device 600 of FIG. 6 with the cam closing mechanism 614 in a closed position, in accordance with at least one example. The actuator 628 has been moved to rotate the cam 630, causing the cam to push the tissue engager 618 in a closing direction 636. Movement of the tissue engager 618 in the dosing direction 636 also moves a portion of the tissue 604 in the movement direction 636 to close the wound 602. Further, when the actuator 628 is moved far enough, the locking feature 650 of the cam 630 engages the corresponding locking feature 652 of the tissue engager 618. The locking features 651, 652 in engagement prevent the cam 630 from moving, which maintains the tissue engager 618 in the closed position.

In the illustrated example, a second tissue engager 638 can be seen through the window 610 having moved toward the wound 602 in the closing direction 636 on the side of the wound closure device 600 that does not include the cutaway plane 616. In some examples, the second tissue engager 638 can be moved by a similar closing mechanism 614. In some examples, the first and second tissue engagers 618, 638 are moved such that they meet to close the wound 602. In at least one example, the wound closure device 600 does not include the second tissue engager 638, and the first tissue engager 618 moves the tissue 604 far enough to close the wound 602. In at least one example, the tissue engager 618 moves completely over the wound 602. In some examples, the tissue engager 618 moves over and beyond the wound 602.

FIG. 8 is a cutaway top view of a wound closure device 800 with a push tab closing mechanism 814 in an open position, in accordance with at least one example. In the illustrated example, the wound closure device 800 includes a housing 808, a dosing mechanism 814, and a tissue engager 818. The cutaway plane 816 is shown with dotted lines, such that the top of the housing 808 is only cut away on one side of the wound closure device 800. The housing 808 can be of any shape. In the illustrated example, the housing 808 includes a window 810 that allows for visibility of the wound 802 and in some examples can accommodate a tube. Some examples may not include a window 810.

The closing mechanism 814 is coupled to the housing 808 and the tissue engager 818, such that the closing mechanism 814 can move the tissue engager 818. A surface of the tissue engager 818 that comes in contact with the tissue includes an engaging feature, for example, adhesive, hooks, teeth, grip, a combination of these, or the like to grab or otherwise engage the tissue. The tissue engager 818 may be smaller or larger than illustrated and may comprise any of a variety of shapes.

In the illustrated example, the push tab closing mechanism 814 includes a push tab actuator 828 coupled to the tissue engager 818, such that the push tab actuator 828 can be pushed to close the closing mechanism 814. In some examples, the push tab actuator 828 can be pulled to open the closing mechanism 814, In some examples, the push tab closing mechanism 815 includes a locking feature 852 that interacts with a corresponding locking feature 850 of the actuator 828 to lock the closing mechanism 814 in the open or closed position. In the illustrated example, the locking feature 862 includes a compression spring and a tab that corresponds with a hump 850 of the push tab actuator 828, such that the closing mechanism 814 can be locked in the open position or the closed position. In some examples the closing mechanism can only be locked in the open position or only in the closed position. In the illustrated example, the lock can be overcome by an external force applied to the push tab actuator 828, such as a pushing force to overcome the open position and a pulling force to overcome the closed position. In other examples, the dosing mechanism 814 may include any of a variety of locking features. In other examples, more or less locking features 850, 852 may be used.

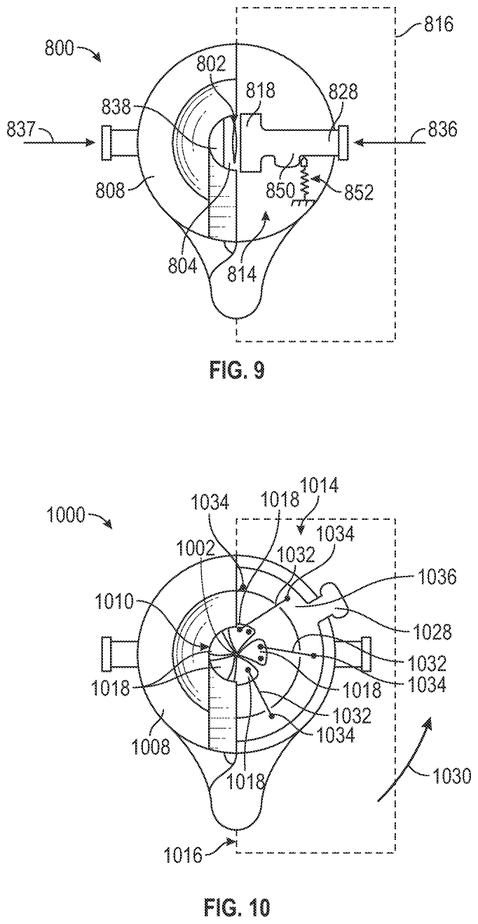

FIG. 9 is a cutaway top view of the wound closure device 800 of FIG. 8 with the push tab closing mechanism 814 in a dosed position, in accordance with at least one example. The actuator 828 has been pushed in a closing direction 836 (toward the wound 802) to move the tissue engager 818 to a closed position. Movement of the tissue engager 618 in the closing direction 836 also moves a portion of the tissue 804 in the closing direction 836 to close the wound 802. Further, when the actuator 828 is pushed far enough, the locking feature 850 of the actuator 818 engages the corresponding locking feature 852, which prevents the push tab actuator from moving in a direction opposite the closing direction 836 and maintains the tissue engager 818 in the closed position.

In the illustrated example, a second tissue engager 838 can be seen through the window 810 having moved toward the wound 802 by movement of a second push tab actuator 848 in the opposite closing direction 837 on the side of the wound closure device 800 that does not include the cutaway plane 816. In some examples, the first and second tissue engagers 818, 838 are moved such that they meet to close the wound 802. In at least one example, the wound closure device 800 does not include the second tissue engager 838, and the first tissue engager 818 moves the tissue 804 far enough to close the wound 802. In at least one example, the tissue engager 818 moves completely over the wound 802. In some examples, the tissue engager 818 moves over and beyond the wound 802.

FIG. 10 is a cutaway top view of a wound closure device 1000 with an iris closing mechanism 1014 in a closed position, in accordance with at least one example. In the illustrated example, the wound closure device 1000 includes a housing 1008, a closing mechanism 1014, and a tissue engager 1018. The cutaway plane 1016 is shown with dotted lines, such that the top of the housing 1008 is only cut away on one side of the wound closure device 1000, The housing 1008 can be of any shape. In the illustrated example, the housing 1008 includes a window 1010 that allows for visibility of the wound 1002 and in some examples can accommodate a tube. Some examples may not include a window 1010.

The closing mechanism 1014 is coupled to the housing 1008 and the tissue engager 1018, such that the closing mechanism 1014 can move the tissue engager 1018. A surface of the tissue engager 1018 that comes in contact with the tissue includes an engaging feature, for example, adhesive, hooks, teeth, grip, a combination of these, or the like to grab or otherwise engage the tissue. The tissue engager 1018 may be smaller or larger than illustrated and may comprise any of a variety of shapes.

In the illustrated example, the tissue engager 1018 includes a plurality of tissue engaging components that form an iris closure. The iris closing mechanism 1014 includes a plurality of rods 1032, a plurality of pins 1034, a disk 1036, and an actuator 1028. Each rod 1032 extends from a component of the tissue engager 1018 to a pin 1034 that is attached to a disk 1036. Movement of the actuator in a closing direction 1030 rotates the disk 1036, which moves each pin 1034, which moves the corresponding rod 1032, which causes the corresponding component of the tissue engager 1018 to move, such that all of the components of the tissue engager 1018 move toward one another to close the wound 1002. In at least one example the iris closing mechanism 1014 includes a locking feature which may lock the closing mechanism 1014 in the open position, the closed position, or both. Throughout the various examples described herein, any of a variety of locking features may be used that take advantage of, for example, pins, tabs, biasing elements, breakaway elements, barriers, a combination of these, or the like.

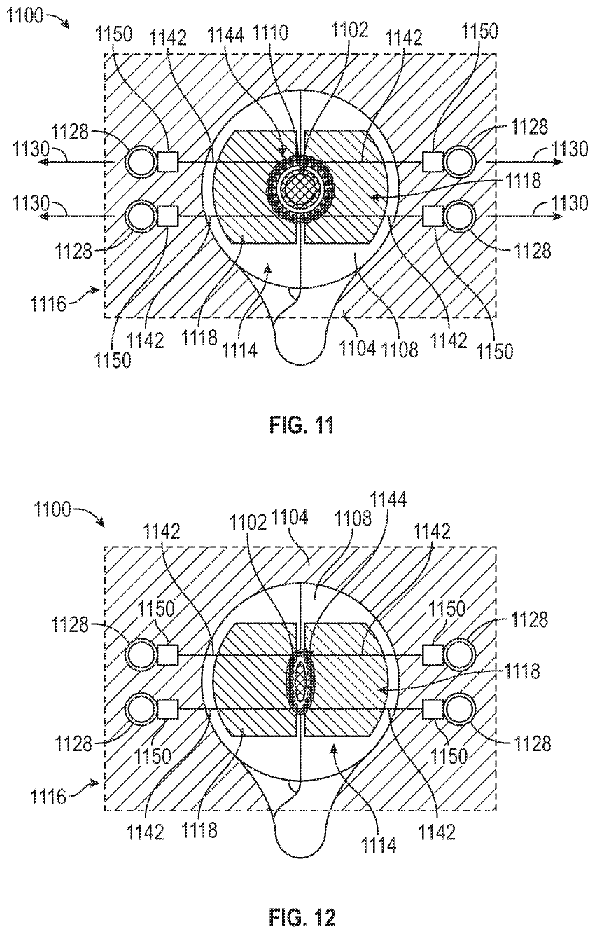

FIG. 11 is a cutaway top view of a wound closure device 1100 with a pull closing mechanism 1114 in an open position, in accordance with at least one example. In the illustrated example, the wound closure device 1100 includes a housing 1108, a dosing mechanism 1114, and a tissue engager 1118. The cutaway plane 1116 is shown with dotted lines, such that the top of the housing 1108 is cut away from the entire wound closure device 1100. The housing 1108 can be of any shape. In at least one example, the wound closure device 1100 does not include the housing 1108. In the illustrated example, the housing 1108 includes a window 1110 that allows for visibility of the wound 1102 and in some examples can accommodate a tube. Some examples may not include a window 1110.

In at least one example, the closing mechanism 1114 is coupled to the housing 1108 and the tissue engager 1118, such that the closing mechanism 1114 can move the tissue engager 1118. A surface of the tissue engager 1118 that comes in contact with the tissue includes an engaging feature, for example, adhesive, hooks, teeth, grip, a combination of these, or the like to grab or otherwise engage the tissue. The tissue engager 1118 may be smaller or larger than illustrated and may comprise any of a variety of shapes.

In the illustrated example, the pull closing mechanism 1114 includes actuators 1128, cables 1142, and a sleeve 1144. The cables 1142 may comprise string, plastic, metal, a combination of these, or the like. In the illustrated example, the actuators 1128 are rings, but in other examples the actuators 1128 could be tabs, handles, or other pull mechanisms. The actuators 1128 can be pulled in the closing direction 1130 to cinch or otherwise tighten the sleeve 1144 which is coupled to the tissue engager 1118. In at least one example, the actuators 1128 tighten the sleeve 1144 on the opposite side of the wound 1102. In at least one example, the sleeve 1144 is woven or includes a woven portion. The sleeve 1144 can be made of cloth, adhesive strip, plastic, a combination of these, or the like. In at least one example, the sleeve 1144 is an adhesive strip (that may not be shaped and may not function as a sleeve). In at least one example, the cables 1142 are pulled to cinch or otherwise move the adhesive strip 1144 to pull the skin.

While the illustrated example shows four actuators 1128, four cables 1142 two tissue engagers 1118, and two woven sleeves 1144, other examples may include more or less of any of these components. In at least one example, a single woven sleeve 1144 extends around at least a portion of the wound 1102 such that in the closed position, the single woven sleeve 1144 causes the one or more tissue engagers 1118 to cinch around the wound 1102 to close the wound 1102. In at least one example, a single actuator 1128 is used per woven sleeve 1144, such that the cable 1142 extends through the woven sleeve 1144 with one end of the cable 1142 coupled to the woven sleeve 1144 and the other end of the cable 1142 is coupled to the actuator 1128 pulling the actuator 1128 cinches the sleeve (or adhesive strip) 1144 about the cable 1142.

FIG. 12 is a cutaway top view of the wound closure device 1100 of FIG. 11 with the pull closing mechanism 1114 in a closed position, in accordance with at least one example. The actuators 1128 have been pulled in the closing directions 1130, tightening the woven sleeves 1144 which are coupled to the tissue engagers 1118 and cause the tissue engagers 1118 to cinch the tissue 1104 together to close the wound 1102. In the illustrated example, locking features 1150 are coupled to the actuators 1128 or the cables 1142. In at least one example the locking feature 1150 includes an adhesive that can be secured to the tissue 1104 to maintain the tension on the cables 1142 to lock the tissue engager 1118 in the closed position. In at least one example, the wound closure device 1100 can be unlocked by detaching the locking feature 1150 from the tissue 1104.

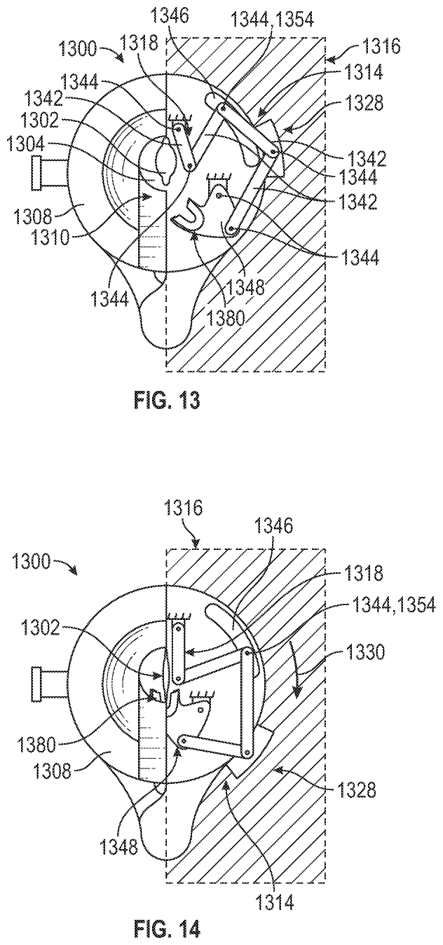

FIG. 13 is a cutaway top view of a wound closure device 1300 with a closing mechanism 1314 in an open position and a tissue fastener 1380 undeployed, in accordance with at least one example. In the illustrated example, the wound closure device 1300 includes a housing 1308, the closing mechanism 1314, a tissue engager 1318, and the tissue fastener 1380. The cutaway plane 1316 is shown with dotted lines, such that the top of the housing 1308 is only cut away on one side of the wound closure device 1300. The housing 1308 can be of any shape. In the illustrated example, the housing 1308 includes a window 1310 that allows for visibility of the wound 1302 and in some examples can accommodate a tube. In some examples the housing 1308 does not include a window 1310.

The closing mechanism 1314 is coupled to the housing 1308 and the tissue engager 1318, such that the closing mechanism 1314 can move the tissue engager 1318. A surface of the tissue engager 1318 that comes in contact with the tissue 1304 includes an engaging feature, for example, adhesive, hooks, teeth, grip, a combination of these, or the like to grab or otherwise engage the tissue 1304. In some examples, the tissue engager 1318 may be smaller or larger than illustrated and may comprise any of a variety of shapes. In the illustrated example, the closing mechanism 1314 includes an actuator, a plurality of connectors 1342, a plurality of hinge points 1344, a pin 1354, a slot 1346, and a tissue fastener holder 1348.

FIG. 14 is a cutaway top view of the wound closure device 1300 of FIG. 13 with the closing mechanism 1314 in a closed position and the tissue fastener 1380 deployed, in accordance with at least one example. In the illustrated example, the actuator 1328 has been moved in the closing direction 1330, such that the pin 1354 moved along the slot 1346, and the connectors 1342 pivot about their respective hinge points 1344 to move the tissue engager 1318, and therefore a portion of the tissue 1304, toward the wound 1302 to close the wound. Additionally, as the connectors 1342 pivot about their hinge points 1344, the tissue fastener holder 1348 pivots about its hinge point to deploy the tissue fastener 1380 to fasten the closed wound 1302 shut.

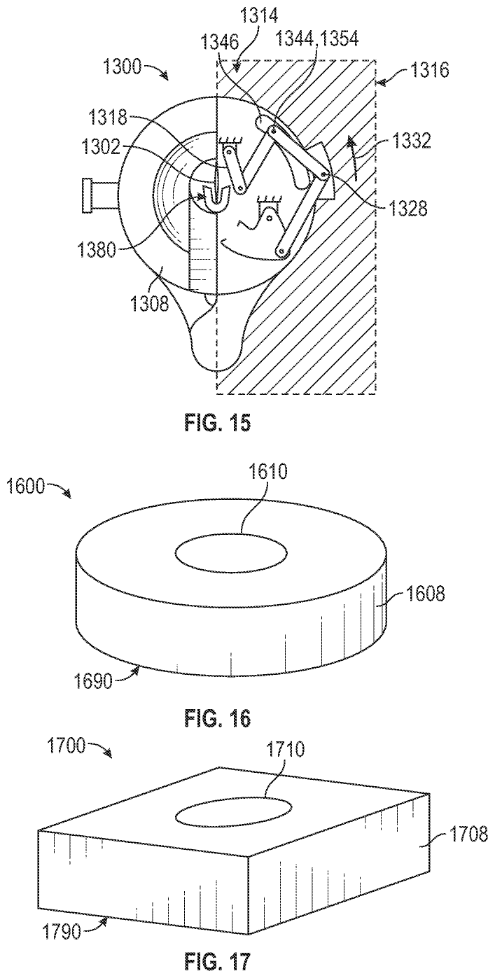

FIG. 15 is a cutaway top view of the wound closure device 1300 of FIGS. 13 and 14 with the dosing mechanism 1314 returned to an opened position and the tissue fastener 1380 deployed, in accordance with at least one example. In the illustrated example, the actuator 1328 has been moved in the opening direction 1332, such that the pin 1354 moved along the slot 1346, and the connectors 1342 pivot about their respective hinge points 1344 to move the tissue engager 1318 and the tissue fastener holder 1348 away from the wound 1302. Even though the wound closure device 1300 is in an open position, the tissue fastener 1380 remains fastened to the wound 1302, such that the wound 1302 remains closed. In at least one example, the wound closure device 1300 may then be removed from the tissue 1304, and the wound 1302 would remain closed by the tissue fastener 1380. In at least one example, the wound 1302 could be closed and the tissue fastener 1380 deployed using one hand. In at least one example, the wound 1302 could be closed and the tissue fastener 1380 deployed in one motion.

In the illustrated example, the tissue fastener 1380 is U-shaped or C-shaped with two prongs and a mating feature that allows it to mate with the tissue fastener holder 1348. In other examples, the tissue fastener 1380 may have more or less prongs. In some examples, the tissue fastener 1380 is biased closed, and the tissue fastener holder 1348 holds the tissue fastener 1380 in an open position, such that once the tissue fastener 1380 is deployed it closes around the wound 1302. The tissue fastener 1380 could comprise any of a variety of materials, for example, plastic, metal, a dissolvable material, a bioabsorbable material, a combination of these, or the like. In at least one example, the tissue fastener 1380 could keep the wound 1302 closed until it healed, and then the tissue fastener 1380 could dissolve or be absorbed.

FIGS. 16 and 17 are perspective views of wound closure devices 1600 and 1700, respectively. In the illustrated examples, the wound closure devices 1600, 1700 include a housing 1608, 1708 that has a window 1610, 1710. The housing 1608, 1708 may be any of a variety of shapes and sizes. In some examples the housing 1608, 1708 may comprise multiple components that are hinged or otherwise coupled together. In at least one example, the housing 1608, 1708 is a single component of unitary construction.

The window 1610, 1710 may be any of a variety of shapes and sizes. In some examples, the window can accommodate a tube, such as a tube implanted in a patient. In at least one example, the window 1610, 1710 is an opening through which the wound can be accessed or viewed. In at least one example the window 1610, 1710 is covered by a transparent or semitransparent material so the wound can be viewed. In at least one example, the window includes a seal. In some examples, a bottom surface 1690, 1790 of the housing 1608, 1708 includes an adhesive or is an adhesive that attaches the wound closure device 1600, 1700 to the tissue.

In at least one example, the housing 1608, 1708 is configured to be handheld in a single hand. In at least one example, the housing 1608, 1708 is no more than three times the size of the wound that needs to be closed. In some examples, the housing 1608, 1708 is less than six inches in diameter (or in each dimension). In at least on example, the housing 1608, 1708 is less than five inches in diameter (or in each dimension).

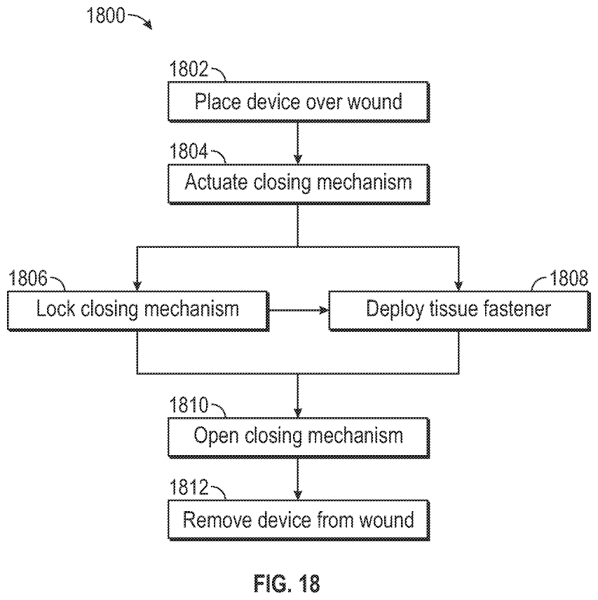

FIG. 18 is a is a flow chart of a method 1800 for closing a wound, in accordance with at least one example. At block 1802, the wound closure device is placed over the wound. In at least one example, the wound closure device is placed over the wound such that the wound is visible through a window of the housing. In some examples, an adhesive or other coupling mechanism allows the wound closure device to attach to the tissue of the patient. In at least one example, placing the wound closure device over the wound causes the tissue engager to engage a portion of the tissue. In at least one example, the wound closure device is placed about a tube implanted in and extending from a patient's body.

At block 1804, the dosing mechanism of the wound closure device is actuated. The dosing mechanism can be any of a variety of dosing mechanisms as described above with reference to FIGS. 1-17 and can be actuated in any of a variety of ways. Actuation of the dosing mechanism moves the tissue engager from an open position to a closed position. As such, in at least one example, actuation of the closing mechanism moves the tissue engager (and as a result at least a portion of the tissue) to close the wound. In at least one example, actuation of the closing mechanism causes the tissue engager to engage a portion of the tissue, In at least one example, the closing mechanism is closed as a tube extending through the wound closure device is removed from a patient's body. In at least one example, the closing mechanism is closed following removal of a tube extending through the wound closure device from a patient's body. In some examples, removal of an implanted tube from at least a portion of the wound closure device causes the closing mechanism to close. In at least one example, the closing mechanism is biased closed. Following actuation of the closing mechanism, the method 1800 can proceed to block 1806 at which the closing mechanism is locked, or to block 1808 at which a tissue fastener is deployed, depending on the embodiment,

At block 1806, the closing mechanism is locked, In at least one example, this secures the tissue engager in the closed position to keep the wound closed. In at least one example that includes a biasing element, the method 1800 does not include block 1806 because the biasing element is strong enough to maintain the closure of the wound. In some examples, the wound closure device can be sealed to avoid infection of the site. Following locking of the closing mechanism at block 1806, the method 1800 can proceed to block 1808 at which a tissue fastener is deployed, or to block 1810 at which the closing mechanism is opened (and unlocked if it was locked).

At block 1808 a tissue fastener is deployed to fasten the closed wound. For example, the closing mechanism may deploy the tissue fastener. In at least one example, the same actuator is used to deploy the tissue fastener as was used to close the closing mechanism. In some examples, a separate actuator may be used to deploy the tissue fastener.

At block 1810, the closing mechanism is opened. In at least one example, the closing mechanism is moved from the closed position to the open position (or to a different open position than the starting open position). If the closing mechanism had previously been locked, then the closing mechanism is unlocked and opened. In some examples where a tissue fastener has been deployed, the tissue fastener remains fastened to the closed wound when the closing mechanism is opened. In some examples the same actuator is used to close the closing mechanism as to open the closing mechanism. In at least one example, a different actuator is used to open the closing mechanism. In at least one example, the closing mechanism is opened to provide access to the wound.

At block 1812, the wound closure device is removed from the wound. In at least one example, the wound closure device may be removed to allow for other procedures, for example suturing, stapling, surgery, cleaning, a combination of these, or the like. In at least one example where a tissue fastener was used, the wound closure device is removed but the tissue fastener remains keeping the wound closed. In at least one example, the wound closure device is removed when the wound has healed or dissolves.

Each of these non-limiting examples can stand on its own or can be combined in various permutations or combinations with one or more of the other examples. The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls with regard to subject matter taught in this document, In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Use of the word “seal” can mean a seal or barrier sufficient to prevent foreign objects (e.g., germs) from entering the incision site or wound surrounded by the device. For example, a “seal” could limit airflow sufficient to avoid interference by foreign bodies that could lead to infection. In some examples “seal” can mean a substantially airtight seal. The tissue engager must engage the tissue with sufficient strength such that movement of the tissue engager will also move the tissue to dose the wound. In at least one example, the tissue engager should have enough force to pinch and hold the tissue in place. In at least one example, the tissue engager should also have enough grip to handle some moderate tissue movement to avoid the tissue engager coming loose at some point during treatment. In at least one example, the tissue engager penetrates a surface of the tissue. In some examples, the tissue engager may comprise plastic, metal, bioabsorbable materials, dissolvable materials, a combination of these, or the like.

While various examples show housings of a particular shape or with specific features, such as a hinge portion, in some examples the housing is any of a variety of shapes and sizes. In some examples, a bottom surface of the housing includes an adhesive or is an adhesive that attaches the wound closure device to the tissue of the patient. Some examples may include more hinge portions or no hinge portion at all. Further, in some examples the housing comprises more than two components or consists of a single component of unitary construction. Some examples with multiple components include a coupling mechanism to couple the various components of the housing together, Further, while some of the illustrated examples can accommodate a tube, such as an implanted tube. In at least one example, the housing is a tube clamp. In some examples, the wound closure device is part of a tube anchoring device. In some examples the housing is not configured to accommodate a tube.

In some examples, the house can include a window of any size and shape. In some examples the window may be covered by a transparent or semitransparent material. In some examples the window may be an opening. In some examples with a window in the form of an opening, the window can later be sealed. While the illustrated examples show a generally round wound, the wound closure devices disclosed herein could similarly be used for wounds of various shapes and sizes,

While the various examples show different arrangements of closing mechanisms, in some examples more or less components may be included. Additionally, more or less of any individual component shown may be provided in some examples. Further, in some examples, two or more of the dosing mechanisms are combined in a single wound closure device. While all wound closure devices include at least one tissue engager, some examples may include more or less tissue engagers than is shown in a given figures. Additionally, in some examples the orientation or position of various components of the closing mechanism and the tissue engager may be different than what is shown and still be within the scope of the present teachings. Further, in some examples, the actuator can comprise any of a variety of handles or shafts. While not shown on in all illustrated examples, any of the illustrated examples can include a lock. Further, in some examples, a lock other than what is shown may be provided. For example, instead of the lock shown in FIGS. 6 and 7, a lock may be provided separate from the cam. In some examples the lock may be activated using the same actuator as the closing mechanism, while in other examples, the lock may be activated using a different actuator than the closing mechanism.

Other non-limiting example configurations are described in the following individually numbered Examples.

Example 1 is a device, comprising: a housing configured to be placed over a wound of a patient; a tissue engager configured to engage a portion of tissue proximate the wound; and a closing mechanism coupled to the housing and the tissue engager, the closing mechanism configured to move the tissue engager from an open position to a closed position to close the wound.

In Example 2, the subject matter of Example 1 optionally includes an adhesive coupled to the housing, the adhesive configured to couple the housing to the patient.

In Example 3, the subject matter of any one or more of Examples 1-2 optionally include wherein the housing defines a window configured for visibility of the wound.

In Example 4, the subject matter of any one or more of Examples 1-3 optionally include wherein the housing is configured to accommodate a tube implanted in and extending from the patient.

In Example 5, the subject matter of any one or more of Examples 1-4 optionally include wherein the dosing mechanism comprises a slider-crank mechanism.

In Example 6, the subject matter of any one or more of Examples 1-5 optionally include wherein the dosing mechanism comprises a dual slider-crank mechanism.

In Example 7, the subject matter of any one or more of Examples 1-6 optionally include wherein the dosing mechanism comprises a cam mechanism.

In Example 8, the subject matter of any one or more of Examples 1-7 optionally include wherein the dosing mechanism comprises a push tab.

In Example 9, the subject matter of any one or more of Examples 1-8 optionally include wherein the dosing mechanism comprises an iris mechanism,

In Example 10, the subject matter of any one or more of Examples 1-9 optionally include wherein the dosing mechanism comprises a pull mechanism.

In Example 11, the subject matter of any one or more of Examples 1-10 optionally include a tissue fastener configured to fasten the dosed wound.

In Example 12, the subject matter of Example 11 optionally includes wherein the dosing mechanism is configured to deploy the tissue fastener.

Example 13 is a device comprising: a housing including a window, the housing configured to be placed over a wound such that the wound can be viewed through the window; at least one tissue engager configured to engage a portion of tissue proximate the wound; and a dosing mechanism coupled to the housing and the tissue engager, the closing mechanism configured to be actuated to move the at least one tissue engager into a closed position to close the wound.

In Example 14, the subject matter of Example 13 optionally includes wherein: the at least one tissue engager comprises first and second tissue engagers, and the closing mechanism is configured to move the first tissue engager toward the second tissue engager to close the wound.

In Example 15, the subject matter of any one or more of Examples 13-14 optionally include wherein the housing is configured to be positioned around a tube implanted in and extending from a patient.

Example 16 is a method comprising: placing a wound closure device over a wound, the wound closure device comprising a housing, a tissue engager, and a closing mechanism; actuating the closing mechanism to move the tissue engager to a closed position, such that the tissue engager causes tissue to close the wound.

In Example 17, the subject matter of Example 16 optionally includes wherein placing the wound closure device over the wound comprises engaging a portion of tissue.

In Example 18, the subject matter of any one or more of Examples 16-17 optionally include locking the closing mechanism in the closed position.

In Example 19, the subject matter of any one or more of Examples 16-18 optionally include deploying a tissue fastener, via the wound closure device, to fasten the wound in the closed position.

In Example 20, the subject matter of Example 19 optionally includes removing the wound closure device from the wound while the tissue fastener remains fastened to the wound in the dosed position,

In Example 21, the subject matter of any one or more of Examples 16-20 optionally include wherein actuating the closing mechanism comprises actuating the closing mechanism with a single hand.

In Example 22, the subject matter of any one or more of Examples 16-21 optionally include wherein actuating the closing mechanism comprises a spring release or a breakaway action.

In Example 23, the subject matter of any one or more of Examples 16-22 optionally include wherein actuating the closing mechanism is automatic following removal of an implanted tube from the wound.

In Example 24, the subject matter of any one or more of Examples 1-23 optionally include wherein the tissue engager includes at least one tissue fastener.

In Example 25, the subject matter of any one or more of Examples 1-24 optionally include wherein the tissue engager comprises a bioabsorbable material.

In Example 26, the subject matter of any one or more of Examples 1-25 optionally include wherein the housing is configured to be coupled to the patient via sutures.

In Example 27, the subject matter of any one or more of Examples 1-26 optionally include wherein the closing mechanism is molded or cast as one or more unitary components.

In Example 28, the subject matter of any one or more of Examples 1-27 optionally include wherein the closing mechanism is actuated via a breakaway mechanism.

In Example 29, the subject matter of any one or more of Examples 1-28 optionally include wherein the dosing mechanism is actuated via a release tab.

In Example 30, the subject matter of any one or more of Examples 1-29 optionally include wherein the dosing mechanism is actuated via release of a biasing element.

In Example 31, the subject matter of any one or more of Examples 11-30 optionally include wherein the tissue fastener comprises a bioabsorbable material.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations.

What is claimed is:

1. A device, comprising:
a housing configured to be placed over a wound of a patient;
a tissue engager configured to engage a portion of tissue proximate the wound; and
a closing mechanism coupled to the housing and the tissue engager, the closing mechanism configured to move the tissue engager from an open position to a closed position to close the wound.

2. The device of claim 1, further comprising:
an adhesive coupled to the housing, the adhesive configured to couple the housing to the patient.

3. The device of claim 1, wherein the housing defines a window configured for visibility of the wound.

4. The device of claim 1, wherein the housing is configured to accommodate a tube implanted in and extending from the patient.

5. The device of claim 1, wherein the closing mechanism comprises a slider-crank mechanism,

6. The device of claim 1, wherein the closing mechanism comprises a dual slider-crank mechanism.

7. The device of claim 1, wherein the closing mechanism comprises a cam mechanism.

8. The device of claim 1, wherein the dosing mechanism comprises a push tab.

9. The device of claim 1, wherein the closing mechanism comprises an iris mechanism.

10. The device of claim 1, wherein the closing mechanism comprises a pull mechanism.

11. The device of claim 1, further comprising:
a tissue fastener configured to fasten the closed wound.

12. The device of claim 11, wherein the closing mechanism is configured to deploy the tissue fastener.

13. A device comprising:
a housing including a window, the housing configured to be placed over a wound such that the wound can be viewed through the window;
at least one tissue engager configured to engage a portion of tissue proximate the wound; and
a closing mechanism coupled to the housing and the tissue engager, the closing mechanism configured to be actuated to move the at least one tissue engager into a closed position to close the wound.

14. The device of claim 13, wherein:
the at least one tissue engager comprises first and second tissue engagers, and
the dosing mechanism is configured to move the first tissue engager toward the second tissue engager to dose the wound.

15. The device of claim 13, wherein the housing is configured to be positioned around a tube implanted in and extending from a patient.

16. A method comprising:
placing a wound closure device over a wound, the wound closure device comprising a housing, a tissue engager, and a dosing mechanism;
actuating the dosing mechanism to move the tissue engager to a dosed position, such that the tissue engager causes tissue to dose the wound.

17. The method of claim 16, wherein placing the wound closure device over the wound comprises engaging a portion of tissue.

18. The method of claim 16, further comprising:
locking the dosing mechanism in the dosed position.

19. The method of claim 16, further comprising:
deploying a tissue fastener, via the wound closure device, to fasten the wound in the closed position.

20. The method of claim 19, further comprising:
removing the wound closure device from the wound while the tissue fastener remains fastened to the wound in the closed position.
